(19) United States
(12) Patent Application Publication
(10) Pub. No.: US 2010/0241202 A1
(43) Pub. Date: Sep. 23, 2010

(54) Zapper with alligator clips

(76) Inventor: Antonio Francesco Carducci, Wimberley, TX (US)

Correspondence Address: Antonio Francesco Carducci, 1213 Ridge Oak Drive, Wimberley, TX, 78676, US

(21) Appl. No.: 12/072,251

(22) Filed: Mar. 23, 2009

(51) Int. Cl. A61N 1/00

(52) U.S. Cl. 607/115

(57) ABSTRACT

Alligator clips are an improvement to Hulda Clark hand holds in a number of ways. Hand holds are a confinement to the hands for as long as the zapper is used. Alligator clips keep the hands free for use during zapper treatment. Alligator clips are attached to wet clothing. The treatment results are clearly visible on the surface of the body and this becomes an encouragement to continue treatment. With the Hulda Clark hand holds the healing energy is applied only to the interior of the body. With alligator clips the application of healing energy reaches the entire body both exterior and interior. With alligator-clips it is possible to see the dead parasites when the treatment is concluded. With the Hulda Clark hand holds the dead parasites are not visible.

My invention is an improvement of a home health care device called Zapper described by Dr. Hulda Clark in a book published in 1995 titled The Cure For All Diseases. The Zapper is a small box containing components, which produces a positive voltage of electricity that kills large and small invaders such as flukes, round worms, mites, bacteria, viruses and fungi. The box is about 4 inches by 2 inches by ½ inch. Two wires are attached to the box. The ends of both wires are attached to two copper pipes, 4 by ½ inches long. The pipes are covered by cloth, which when wet conduct electric current. The electric current passes from the palm of the hands to the blood stream of a person and from the bloodstream to every cell in the body where it kills the parasites. The zapper has not been patented. The zapper has not been presented to the FDA for approval.

My invention replaces the handholds with two alligator clips. Cloth more conveniently clothing, is attached to the alligator clips. The cloth or clothing, when wet conducts the electric current to all the exterior surface of the body covered by the wet clothing killing the parasites mentioned above. From the skin the current continues on to the blood stream where it performs the same function as the zapper. With the use of alligator clips conductivity of the current to the blood is much more positive by virtue of the larger surface of skin employed for the purpose. Whereas the zapper with handholds does not kill any parasites on the skin, the zapper with alligator clips kills parasites on the entire surface of the body.

My improvement to the Zapper is a much more positive conductor of positive current to the bloodstream. It is possible to introduce the electric current to the entire body at one time multiplying the amount of current to the interior.

1. The Hulda Clark Zapper introduces energy to the interior of the body only through the palms of the hands there by killing parasites in the interior of the body only.

2. The Hulda Clark zapper ties up the hands so that the hands are unable to perform any other function during the time of treatment

3. My improvement to the Hulda Clarck zapper hereinafter referred to as improved Zapper uses alligator clips instead of tubing which my be attached to any wet article that conducts energy leaving the hands available for other activities

4. My improved zapper is capable of introducing energy to the entire surface of the body which energy is able to kill parasites on the wet skin, hair and fingernails.

5. My improved Zapper is a more positive provider of energy to the interior of the body because energy is introduced to the interior of the body by the entire surface of body instead of through the few inches of the palm of the hands.

6. My improved zapper creates the very best home health device ever invented by any one in the world by virtue of the healing done to the entire body rather than to the interior only

7. My improved Zapper does a great deal more healing than the Hulda Clark Zapper at a reduced rather than increased cost.

8. My improved Zapper can be used while driving an automobile or while walking or while doing many other tasks.
